(19) United States
(12) Patent Application Publication
(10) Pub. No.: US 2022/0199237 A1
(43) Pub. Date: Jun. 23, 2022

(54) PROCESS TO DEFINE TAILORED INTERVENTION OUTREACH SENT TO PATIENTS DETERMINED TO BE AT RISK OF NO-SHOWING OR CANCELLING LATE TO AN UPCOMING EPISODE OF CARE

(71) Applicant: KONINKLIJKE PHILIPS N.V., Eindhoven (NL)

(72) Inventors: Sara GARVER, CAMBRIDGE, MA (US); Rohit SINGH, CAMBRIDGE, MA (US); Amanda BAKERLEE, CAMBRIDGE, MA (US); Anthony MONFORTE, TEMPE, AZ (US)

(21) Appl. No.: 17/529,337

(22) Filed: Nov. 18, 2021

Related U.S. Application Data
(60) Provisional application No. 63/127,632, filed on Dec. 18, 2020.

(51) Int. Cl. G16H 40/20; G16H 10/60; G06Q 10/06

(52) U.S. Cl. CPC G16H 40/20; G06Q 10/0635; G16H 10/60

(57) ABSTRACT

A patient appointment notification method includes: accessing a scheduler of a medical facility to identify patients scheduled for medical appointments; retrieving patient data for the patients from one or more databases; generating a risk score for one or more patients based on at least the retrieved patient data, the risk score being indicative of a likelihood that the one or more patients will no show or late cancel the medical appointment; and for the one or more patients having a risk score exceeding a predetermined risk score threshold, generating a notification based at least on the risk score exceeding a predetermined risk score threshold and outputting the notification to a device.

### CROSS REFERENCE TO RELATED APPLICATIONS

This application claims the benefit of U.S. Provisional Patent Application No. 63/127,632 filed Dec. 18, 2020. This application is hereby incorporated by reference herein.

### FIELD

The following relates generally to the medical arts, patient appointment scheduling arts, patient appointment notification arts, patient appointment preparation support arts, mobile application arts, and related arts.

### BACKGROUND

Patients often do not show up (i.e., “no show”) for medical appointments, or cancel at a time close to an appointment (i.e., a “late cancellation”). No shows and late cancellations by patients to medical appointments have significant ramifications for both the hospital and the patients. Unused patient appointment time slots and medical device underutilization negatively impact a medical facility's finances and physician's ability to provide effective and timely clinical care. Missed appointments can also negatively impact patient outcomes. No shows for departments like primary care can result in increased emergency department (ED) admissions and a reduction in quality of life for patients.

At the same time, backlogs of to-be-scheduled appointments continue to grow. If appointments such as screenings do not happen within a certain timeframe, reimbursement rates, hospital ratings, and clinical outcomes are also negatively impacted. Supplemental and/or automated processes to assist in growing workloads are necessary to support staff and to keep patients engaged in their care.

The following discloses new and improved systems and methods to overcome these problems and others.

### SUMMARY

In one disclosed aspect, a non-transitory computer readable medium stores instructions executable by at least one electronic processor to patient appointment notification method. The method includes: accessing a scheduler of a medical facility to identify patients scheduled for medical appointments; retrieving patient data for the patients from one or more databases; generating a risk score for one or more patients based on at least the retrieved patient data, the risk score being indicative of a likelihood that the patient will no show or late cancel the medical appointment; and for the one or more patient having a risk score exceeding a predetermined risk score threshold, generating a notification based at least on the risk score exceeding a predetermined risk score threshold and outputting the notification to a device.

In another aspect, a non-transitory computer readable medium stores instructions executable by at least one electronic processor to patient appointment notification method. The method includes: training a machine-learning (ML) component with historical training data including at least historical patient data including historical patient histories of no shows or cancellations; accessing a scheduler of a medical facility to identify patients scheduled for medical appointments; retrieving patient data for the patients from one or more databases; generating a risk score for the patients based on at least the retrieved patient data, the risk score being indicative of a likelihood that the patient will no show or late cancel the medical appointment; and for patients having a risk score exceeding a predetermined risk score threshold, generating a notification based at least on the risk score exceeding a predetermined risk score threshold and outputting the notification to a device.

One advantage resides in reducing patient appointment cancellations and no shows.

Another advantage resides in properly allocating medical facility resources for patient appointments.

Another advantage resides in reducing patient appointment backlogs.

Another advantage resides in providing notifications to patients for upcoming appointments.

Another advantage resides in providing notifications to patients for upcoming appointments that includes traffic information, weather information, financial information, links to preparation resources, and/or confirmation information.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for generating patient appointment notifications in accordance with the present disclosure.

FIG. 2 shows an example flow chart of operations suitably performed by the apparatus of FIG. 1.

FIG. 3 shows an example of a timeline of medical appointment events during which the apparatus of FIG. 1 can be used.

### DETAILED DESCRIPTION

The following relates to patient engagement systems for engaging patients prior to scheduled appointments. Existing systems of this type typically automatically send outreach emails and/or text messages to patients ahead of scheduled medical visits such as scheduled radiology examinations. The messages provide day ahead or other reminders to the patient, and the email or text message may also include links to websites such as explanations of a computed tomography (CT) scan, preparatory instructions, or so forth.

The following relates to improvements designed to reduce the occurrences of no shows or late cancellations (e.g., cancellation within 3 days of the visit, or some other defined late cancellation time frame).

In some embodiments disclosed herein, a first machine learning (ML) component is trained to generate a risk score for the patient indicative of the risk that the patient will no show or late cancel. The risk score is suitably based on inputs such as historical record of the patient (e.g., number of no shows or late cancellations in the last three months, six months, year, et cetera), demographic information (e.g., an elderly patient may be more likely to no show or late cancel; similarly, lower income patients are more likely to no show or late cancel due to transportation issues), referring medical professional (e.g., a patient referred for the examination by an “M.D.” medical doctor may be less likely to no show or late cancel compared with a patient referred by a nurse practitioner or other “lower level” medical professional), the type of examination (e.g., there are statistically more no shows and late cancellations for nuclear medicine imaging examinations compared with magnetic resonance imaging procedures (MRIs) or computed tomography procedures (CTs)), location information (e.g., based on the patient's zip code the travel distance to the medical facility can be estimated and used as an input, longer travel distance typically correlating with higher no shows and late cancellations), and/or so forth. Additional inputs may relate to the specific timing of the examination. For example, examinations scheduled close to a major holiday are more likely to experience no shows or late cancellations. Weather forecasts, seasonal information, and traffic information can be further inputs (e.g., bad weather increases likelihood of no shows or late cancellations, as does the winter seasons and/or the examination being scheduled during traffic rush hour). Additional inputs may relate to patient engagement with resources provided by the medical facility, such as informational websites, preparatory instruction websites, and so forth. Patient engagement inputs could also include whether the patient affirmatively responded to previous reminder emails or text messages or simply ignored them. These are merely illustrative features that may be input to the risk scoring ML component.

While the optimal ML algorithm is expected to be task-dependent, in some initial simulations an Extreme Gradient Boost (XGB) classifier or Lasso Logistic algorithm appears to work well for some risk scoring tasks. The inputs to the risk scoring ML algorithm may be beneficially organized into multiple feature vectors with each feature vector containing features related to a specific cause of no shows or late cancellations. For example, one feature vector may store features relating to past history of no shows or late cancellations (e.g., with individual vector elements being for different time frames). Another feature vector may store features relating to transportation issues, such as distance to the medical facility, estimated income level (possibly estimated from zip code), weather forecast information, and/or so forth. Another feature vector may store features relating to patient engagement, such as features identifying which websites are accessed and/or features indicating when those websites were accessed.

In some embodiments disclosed herein, the risk scoring ML component is suitably trained on historical patient data. The training may also include feature selection, such as performing sensitivity analysis to determine which feature vectors are most probative of no shows or late cancellations. Different risk scoring ML components may be trained for different medical facilities since the populations served by different medical facilities may be different. Likewise, different risk scoring ML components may be trained for different types of examinations (e.g., medical imaging, colonoscopy, etc.). In the currently implemented embodiment, the risk scoring ML component outputs a continuous real-valued risk score in the range [0,1], where 0 indicates a negligible risk of no showing or late cancellation while 1 indicates a high likelihood of no showing or late cancellation.

In the application phase, the trained risk scoring ML component is applied to patients scheduled for examinations. To do this, the risk scoring ML component is integrated into (or at least communicates with) the scheduler used by the medical facility for scheduling examinations, and with information sources such as the Electronic Medical Record (EMR) to extract patient-specific information, the scheduler to extract examination type, National Oceanic and Atmospheric Administration (NOAA) or another weather forecast source, a traffic flow monitoring resource, and/or so forth. The extracted data are preprocessed to generate feature values for the feature vectors. For example, an electronic map may be accessed to estimate travel distance given the patient's zip code and the location of the medical facility. Public databases may be accessed to estimate the patient's income level based on the zip code. The resulting feature vectors for the patient are input to the trained risk scoring ML component to generate the patient's risk score.

In some embodiments disclosed herein, the patient's risk score is the output of the system (e.g., patients whose risk scores are higher than a threshold are flagged in the scheduler for manual follow-up). In a more automated approach, the flagged patients are automatically followed up using automatically generated reminder and/or informational email and/or text messages. Other types of remediation may be performed as well. For example, in the case of appointments such as consultations which have some flexibility in workflow, the system may double-book time slots assigned to patients having high risk scores. Manual and automated follow-up may also be combined. For example, initial automated follow-up in the form of automatically generated reminder and/or informational emails and/or text messages may be initially sent, but if these are ignored by the patient (or, in the case of informational emails, if the patient does not follow up on links to the preparation instructions or the like) then a personal call by a human staffer may be made to the patient.

In other embodiments disclosed herein, a second ML component classifies the source of risk for the flagged patients. For example, if the inputs to the risk scoring ML algorithm are organized into multiple feature vectors with each feature vector containing features related to a specific cause of no shows or late cancellations, then the feature vector or vectors that drove the risk score up for a given patient can be identified, and thereby the corresponding cause is identified. Then, the automatically generated informational emails and/or text messages can be tailored for the specific (likely) cause of the (probable) no show or late cancellation. For example, if the feature vector relating to transportation issues caused the high-risk score then informational emails and/or text messages providing information on transportation options (possibly including links to public transport websites, hospital-provided transportation websites, etc.) can be sent to the patient. On the other hand, if the feature vector relating to patient engagement drove up the risk score then the emails and/or text messages can feature informational sources, possibly presented in different ways. In a more aggressive approach, if the feature vector storing features relating to past history of no shows or late cancellations indicates the patient is a very high risk for no showing or late canceling, then reminder emails may be formulated to indicate that the patient must affirmatively confirm the appointment via email or text message reply or else the appointment will be canceled by the medical facility.

In some embodiments disclosed herein, the risk analysis is performed at a single fixed time, e.g., four days prior to the scheduled examination. In other embodiments disclosed herein, the risk analysis may be performed repeatedly. In this latter approach, the risk score may evolve as the patient approaches the appointment date due to events such as the patient responding (or not responding) to automated reminders, changes in the weather forecast, and/or so forth.

In some embodiments disclosed herein, the system can adaptively update the follow-up based on tracking no shows and late cancellations occurring after the system is put into place. For example, initially the system may send frequent reminder emails and/or text messages to patients at high risk. If the tracking indicates this is ineffective for a particular task (e.g., a particular hospital, a particular cohort of patients, and/or a particular type of examination), then the system may take the more aggressive approach of requiring that the patient must affirmatively confirm the appointment via reply to a reminder email or text message or else the appointment will be canceled by the medical facility. Similarly, patients at risk due to transportation issues may initially receive informational emails about public transportation options; if this is shown to be ineffective by the automatic tracking then the system may provide information about free transportation provided by the hospital itself. This can allow the hospital to limit information on its free transportation (which can be a financial burden on the hospital) to situations where the cost of providing the free transportation will be offset by reduced no shows and late cancellations.

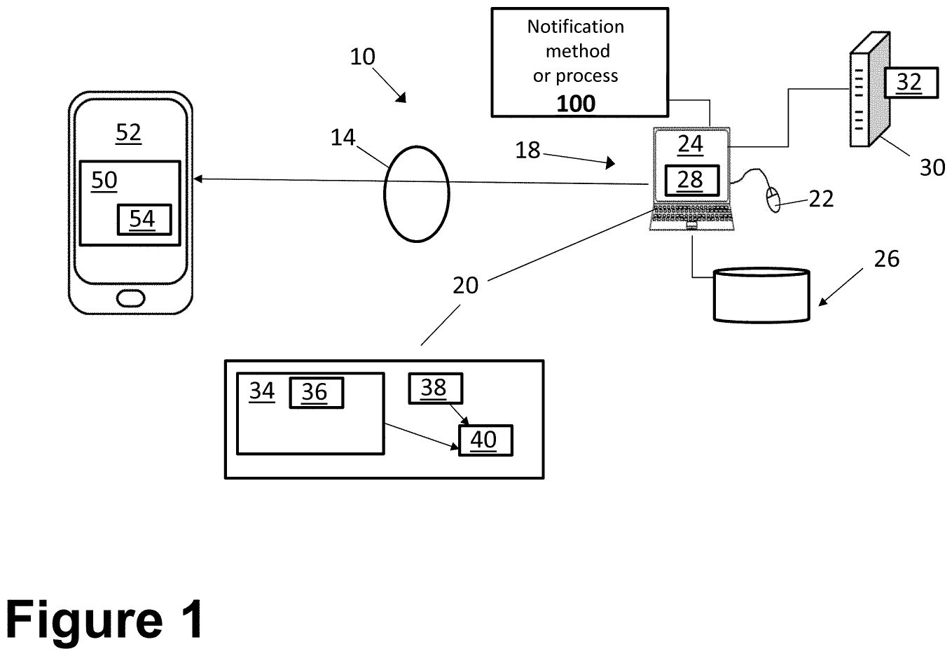

With reference to FIG. 1, an illustrative apparatus 10 is shown for providing patient appointment notifications to patients. FIG. 1 also shows an electronic processing device 18, such as a workstation computer, or more generally a computer. The electronic processing device 18 typically includes or accesses a patient scheduling system, and may also include a server computer or a plurality of server computers, e.g., interconnected to form a server cluster, cloud computing resource, or so forth, to perform more complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the workstation 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The apparatus 10 also includes, or is otherwise in operable communication with, one or more database(s) 30 storing a data related to a queue of patients with schedule medical procedures time and/or data related to operations of the medical facility where the scheduled medical procedures are to occur. The database 30 can be any suitable database, including a Radiology Information System (RIS) database, an Electronic Medical Records (EMR) database, an Electronic Health Records (EHR) database, a Cardiology Information System (CIS) database, a Health-Level 7 (HL7)-compliant database, and so forth. Alternatively, the database 30 can be implemented in the non-transitory medium or media 26. In addition, the database(s) 30 can include a public database storing residence address information for the patients, along with weather information and local traffic information based on the address information of the patient. The residence address information can include, for example, a zip code of the patients, a street address of the patient, or both.

In general, the one or more database(s) 30 include or provide the functionality of: (i) a scheduler 32, which maintains a schedule of appointments for the medical examination thus providing the patient queue; and (ii) a patient data repository such as an EMR or EHR that stores patient data from which the patient features are extracted. As an example of the scheduler 32, a radiology laboratory may have a scheduler component built into the RIS which provides for making patient appointments as well as storing the examination order for each patient. The scheduler 32 typically stores specific appointment times for the patients of a queue of patients, and the order of patients in the queue is then determined from the patient appointment times (e.g., if patients A, B, C, D, and E have respective appointment times of 10:00 am, 10:30 am, 11:00 am, 11:30 am, and 1:00 pm, then the patients are ordered in the queue as A-B-C-D-E where A is the first patient to be seen and E is the last patient to be seen). In practice, it is often not possible to perform every patient examination at the scheduled start time, e.g., if there is only a single medical imaging device and the examination of patient B in the last example runs from 10:30 am to 11:15 am then the examinations of patients C, D, and E will be pushed back and start at times later than their scheduled appointment times. Additionally or alternatively, some or all patients may not have specified appointment times and are then otherwise ordered into the queue. For example, an emergency patient may be “fitted into” the schedule in various ways, such as by being assigned the next available examination slot. It is also contemplated in some environments to have only the queue order without specific patient appointment times, for example as may be done for a laboratory that is servicing only in-hospital patients. In this case, the queue may specify patients to be serviced in the order A-B-C-D-E but without specifying specific appointment times for the patients.

FIG. 1 also shows modules implemented by the at least one electronic processor 20 to generate and send patient appointment notifications. A machine-learning (ML) component 34 (e.g., an Extreme Gradient Boost (XGB) classifier, a Lasso Logistic algorithm, or any other suitable ML algorithm) is configured to generate a risk score 40 indicative of a likelihood that the patient will no show or late cancel the medical appointment. For example, the risk score 40 is scored on a scale of 0 and 1 in which a score of 0 is indicative of a low risk of no show or cancellation and a score of 1 is indicative of a high risk of no show or cancellation. (This is just an example, and other scoring scales can be used; it is also contemplated to rescale and/or discretize the score, e.g., to an integer value between 0 and 10). To do so, the machine-learning algorithm 34 is configured to implement a prediction model 36 to determine which patients are likely to cancel or no show their appointments. In addition, a second ML component 38 (e.g., a cause-classifying ML component) is configured to identifying at least one cause of the risk score 40 if the risk score 40 exceeds a predetermined risk threshold. If, for example, the risk score 40 for a particular patient is 1, then the cause-classifying ML component 38 is configured to identify one or more causes for the risk score being 1 instead of 0.

When a risk score 40 for a particular patient exceeds the predetermined risk threshold, the at least one electronic processor 20 is programmed to generate a notification 50 and transmit the notification 50 to a mobile device 52 of the patient via a communication link 14, which typically comprises the Internet possibly augmented by local area networks. For example, the patient can log-in into a mobile application program (“app”) which is provided as a component of the patient engagement system, and is loaded on, and executable on, the mobile device 52 of the patient (e.g., an illustrative cellular telephone 52, or a tablet computer, personal data assistant or PDA, and/or so forth) to receive the notification 50. The app may be downloaded to the mobile device 52 from an app store (such as Google Play or Apple Store) accessed via a Wi-Fi, cellular, or other wireless communication network. In a suitable embodiment, the app is represented on the home screen or applications screen of the mobile device 52 as an app icon (i.e., a small square, round, or other compact graphical element representing the app) and the user launches (i.e., initiates running of) an instance of the app on the device 52 by touching the icon on a (touch-sensitive) screen of the mobile device 52. In another example, in lieu of the app, the notification 50 can be sent to the mobile device 52 in any suitable format, such as a pre-recorded voice communication, an automated phone call, an email, a short message service (SMS) message, (i.e., a text message), and so forth.

The apparatus 10 is configured as described above to perform a patient appointment notification method or process 100, along with a training operation 101 for training the ML component 34 for generating the risk score 40 for each patient. The non-transitory storage medium 26 stores instructions which are readable and executable by the at least one electronic processor 20 to perform disclosed operations including performing the patient appointment notification method or process 100 and the training method 200. In some examples, the methods 100, 200 may be performed at least in part by cloud processing.

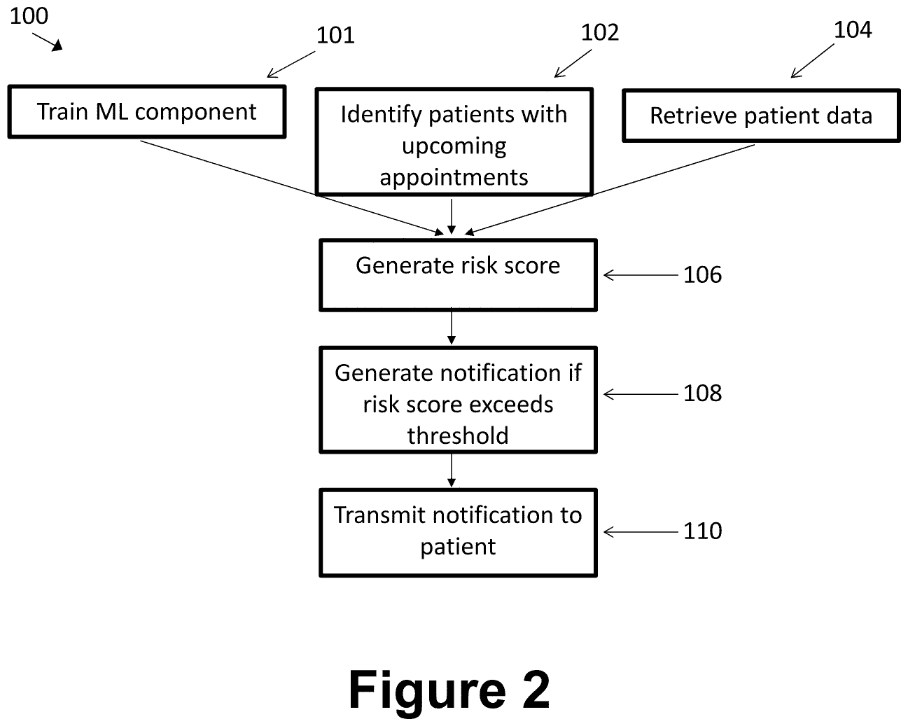

With reference to FIG. 2, an illustrative embodiment of an instance of the patient appointment notification method 100 is diagrammatically shown as a flowchart. The method 100 can begin with the training operation 101 to train the ML component 34 to generate the risk scores 40 (although in some embodiments, the ML component 34 can be pre-trained). To perform the training operation 101, data can be retrieved from the database(s) 30. The retrieved data can include, for example, historical patient data including historical patient histories of no shows or cancellations of appointments for individual patients, patient demographic information (e.g., location, distance from medical facility, payor, gender, vital sign data, age, and so forth), referring physician information, medical examination information that is the subject of the patient appointment (e.g., department, specialty, provider, datetime, etc.), or any combination of these types of data. Machine-learning algorithms are applied to the retrieved data to generate one or more metrics to create the prediction model 36. The ML component 34 is trained on the prediction model 36 (along with updated data which can be input subsequently).

At an operation 102, the scheduler 32 can be accessed to identify patients scheduled for upcoming medical appointments. The accessing of the scheduler 32 can include retrieving, from the scheduler 32, the medical procedure types for the medical appointments of the identified patients. At an operation 104, patient data for the patients schedule for upcoming medical appointments is retrieved from the database(s) 30. In some examples, the retrieved data can include residence address information for the patients and travel difficulty information (such as, for example, at least one of weather forecast information and traffic forecast information), patient history of no shows or late cancellations, and so forth. It will be appreciated that the operation 102, 104 can be performed concurrently or consecutively (in either order).

At an operation 106, the risk score 40 for each patient is generated based on the retrieved data from the operation 104. In some examples, the risk score 40 can be calculated further based on the medical procedure types from the scheduler 32 from the operation 102. In other examples, the risk score 40 can be generated based on the retrieved travel difficulty information and the residence address information of the patient from the operation 104. In some embodiments, the risk score 40 for each patient is generated at a fixed time before the medical appointment of the patient (e.g., 3 or more days). In other examples, the risk score 40 for each patient can be continuously generated or updated.

At an operation 108, a notification 50 can be generated for patients having a risk score exceeding a predetermined risk score threshold (e.g., a score of 1 versus a score of 0). This can be done in a variety of (non-exclusive) examples. The notification 50 can, for example, provide the time of the patient's appointment (e.g., “You have an appointment at (X) medical facility at (X) time.”). In one example, the notification 50 can include a request or prompt 54 for a follow-up phone call or message by the medical facility to the patient having the risk score exceeding the predetermined risk score threshold (e.g., “You have an appointment at (X) medical facility at (X) time. Please call us to confirm”). In another example, the notification 50 can include a request or prompt for the patient to provide an input to confirm the medical appointment. (e.g., “You have an appointment at (X) medical facility at (X) time. Please reply to this message to confirm”). In a further example, the notification 50 can include transportation, weather, and/or financial information based on the patient's location.

In some embodiments, the cause-classifying ML component 38 is configured to identify at least one cause of the risk score exceeding the predetermined risk score threshold (i.e., the patient having a score of 1). For example, the patient may live in an area with high traffic and/or poor weather (which can result in no shows or cancellations by the patient). For example, the patient may be stuck in traffic due to traffic congestion or inclement weather on their way to the appointment. The patient may then call to late cancel the appointment. In another example, the patient may have an income level in which the patient cannot provide payment for the medical appointment. These are merely illustrative examples, and should not be construed as limiting.

To identify the cause(s), one or more feature vectors are generated for specific causes of no shows or late cancellations. The cause-classifying ML component 38 is configured to determine one or more feature vectors most strongly contributes to the risk score 40 exceeding the predetermined threshold. The cause(s) of the no shows/late cancellations are identified as corresponding to the determined feature vector(s). In one particular example, the notification 50 can constitute a cause-specific notification based on the identified cause(s). For example, the notification 50 can say “You have an appointment at (X) medical facility at (X) time. Traffic between your location and the medical facility is heavy during this time. The medical facility provides a shuttle bus service. If you would like to use this service, please call (###) ### #### to schedule your shuttle bus pickup.” Similar messages can include information for other causes, such as weather information or financial information (e.g., providing information for financial aid services). The notification 50 can include such information. In another example. the risk score 40 can be computed dynamically taking into account changes in one or more of patient information, weather information, traffic information, transportation information, and/or social events information.

At an operation 110, the notification 50 is transmitted to the device 52 operable by the patient. In some embodiments, the notification(s) 50 is generated (at the operation 108), and then must be sent to the corresponding patients (e.g., a staff member at the medical facility can control the processing device 18 to send the notifications 50. In other embodiments, the notifications 50 can be automatically transmitted to the device(s) 52. As previously described, the notification 50 can include a prompt or request 54 for the patient to confirm the appointment. The responses can be displayed on the display device 24 of the processing device 18. In some examples, the generating of the risk score 40 is repeated based on a response or non-response to the provided prompt 54. In this example, if the updated risk score exceeds the predetermined risk score threshold, then an updated notification 50 is generated based on the response or non-response to the provided input, and the updated notification 50 is outputted to the device 52. In addition, the updated risk score can be used to update the schedule.

Optionally, the processing of FIG. 2 may be repeated for a given patient multiple times, e.g., on a daily basis as the patient approaches the date of the appointment. The score computed at operation 106 may change from one iteration to the next due to events occurring in the interim. For example, if the patient confirms the appointment at operation 108, then the risk score generated by the next iteration of the method at operation 106 may be lowered due to the confirmation. Conversely, if the patient fails to confirm the appointment at operation 108 (that is, does not send any response at all), then the risk score generated by the next iteration of the method at operation 106 may be increased due to the patient's non-responsiveness which may be indicative of a lack of commitment to make the appointment. The change in score from one iteration to the next of the method of FIG. 2 can result in adjustment of the nature of the notification presented at operation 108. For example, if the patient fails to confirm in two iterations, then the third iteration may indicate that the patient must confirm, or the appointment will be canceled by the medical facility.

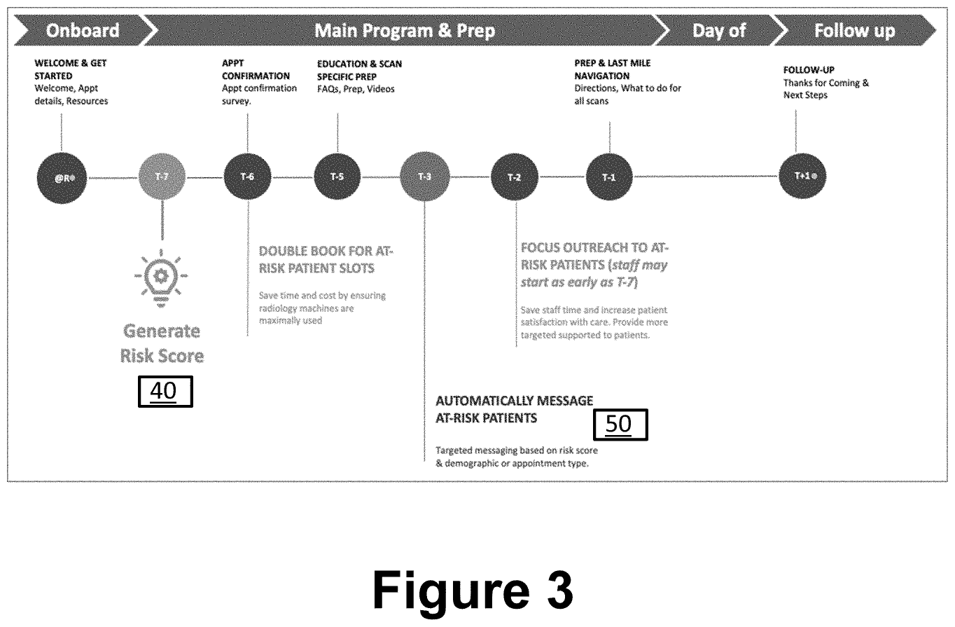

FIG. 3 shows an example of a timeline of events, actions, input, and outputs for a patient medical appointment. Specifically, FIG. 3 shows a timeline for a diagnostic radiology examination. As shown in FIG. 3, there are four periods in the timeline: “onboard”, “main program & prep”, “day of” and “follow up”. In the “onboard” section, a patient welcome, appointment details, and resources for the appointment are determined. In a first event of the “main program & prep” section, the risk score 40 is generated (i.e., the method 100 is performed) at an event labeled “T−7”. Appointment confirmations can be sent at “T−6”. An education and scan specific preparation step can be performed at “T−5”. At “T−3”, the notifications 50 are sent to the patients have a risk score 40 of 1. At “T−2” the more detailed notifications 50 can be sent to the relevant patients. At “T−1”, last minute preparation is performed. The scan is then performed during the “day of” section (i.e., “T=0”), with patient follow up appointments being performed in the “Follow up” section.

In some embodiments, the output of the prediction model 36 implemented by the ML component 34 is used to identify appointments likely to be no-shows or late cancellations. A list of appointments and the respective patients can comprise an initial list for targeted outreach using the notifications 50. Factors, such as proprietary patient specific interaction data, patient demographics, appointment specific factors, and so forth, can be used to make suggestions for target patient groups. These target groups can then be analyzed to assess current outreach modalities and content. Two or three of these target groups can be identified that need to be reviewed in light of client context (e.g., the institution and resources it provides/has available to support at-risk patients). Once the target risk score 40 and components are used to identify the two or three target groups for automated messaging with the notifications 50. The value of the notifications 50 is to reduce no shows and late cancellation rates, which results in higher efficiency of available time slots and medical equipment utilization. Targeting specific groups with the notifications 50 can provide more optimal results.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A non-transitory computer readable medium storing instructions executable by at least one electronic processor to patient appointment notification method, the method comprising:
accessing a scheduler of a medical facility to identify patients scheduled for medical appointments;
retrieving patient data for the patients from one or more databases;
generating a risk score for one or more patients based on at least the retrieved patient data, the risk score being indicative of a likelihood that the one or more patient swill no show or late cancel the medical appointment; and
for patients having a risk score exceeding a predetermined risk score threshold, generating a notification based at least on the risk score exceeding a predetermined risk score threshold and outputting the notification to a device.

2. The non-transitory computer readable medium of claim 1, wherein the method further comprises:
for one or more patients whose risk score exceeds the predetermined risk score threshold, identifying at least one cause of the risk score exceeding the predetermined risk score threshold.

3. The non-transitory computer readable medium of claim 2, wherein the identifying includes:
generating feature vectors for specific causes of no shows or late cancellations;
determining at least one feature vector of the generated feature vectors most strongly contributing to the risk score exceeding the predetermined threshold; and
identify at least one cause of no shows or late cancellations corresponding to the determined at least one feature vector.

4. The non-transitory computer readable medium of claim 3, wherein:
the notification is a cause-specific notification generated further based on the identified at least one cause of no shows or late cancellations.

5. The non-transitory computer readable of claim 2, wherein the identifying of the at least one cause is performed by a cause-classifying machine-learning component.

6. The non-transitory computer readable medium of claim 1, wherein the notification includes:
a request for a follow-up phone call or message by the medical facility to the one or more patients having the risk score exceeding the predetermined risk score threshold.

7. The non-transitory computer readable medium of claim 1, wherein the outputting of the notification includes:
automatically transmitting the notification to the device which is operable by the one or more patients having the risk score exceeding the predetermined risk score threshold.

8. The non-transitory computer readable medium of claim 7, wherein the generating of the notification includes:
including, in the notification, a prompt for the patient receiving the transmitted notification to provide an input to the device operable by the one or more patients to confirm the medical appointment.

9. The non-transitory computer readable medium of claim 8, wherein the generating of a risk score for the one or more patients based on at least the retrieved patient data is repeated to generate an updated risk score for the one or more patients based on the retrieved patient data and further based on a response or non-response to the provided prompt.

10. The non-transitory computer readable medium of claim 9, wherein, if the updated risk score exceeds the predetermined risk score threshold, then:
an updated notification is generated based on the response or non-response to the provided input, and the updated notification is outputted to the device.

11. The non-transitory computer readable medium of claim 9, wherein, if the updated risk score exceeds the predetermined risk score threshold, then:
updating the scheduler of the medical facility based on the updated risk score.

12. The non-transitory computer readable medium of claim 1, wherein:
the accessing of the scheduler further includes retrieving, from the scheduler, the medical procedure types for the medical appointments of the identified one or more patients; and
the risk score for each patient is generated further based on the medical procedure type of the medical appointment for the one or more patients.

13. The non-transitory computer readable medium of claim 1, wherein the method further comprises:
accessing the one or more databases to retrieve residence address information for the one or more patients and travel difficulty information including at least one of weather forecast information and traffic forecast information;
wherein the risk score for the one or more patients is generated further based on the retrieved travel difficulty information and the residence address information of the one or more patients.

14. The non-transitory computer readable medium of claim 13, wherein the outputting further includes:
including, in the notification, information related to one or more of weather forecast information or traffic forecast information based on the residence address information.

15. The non-transitory computer readable medium of claim 1, wherein the retrieved patient data upon which the risk score for the one or more patients is generated includes patient history of no shows or late cancellations.

16. The non-transitory computer readable medium of claim 15, wherein generating a risk score includes:
training a machine-learning component with historical training data including at least historical patient data including historical patient histories of no shows or cancellations;
wherein the risk scores are generated using the trained machine-learning component.

17. The non-transitory computer readable medium of claim 15, wherein the retrieved patient data upon which the risk score for each patient is generated further includes patient demographic information and the generating the risk score includes:
further training the machine-learning component with historical patient demographic information.

18. The non-transitory computer readable medium of claim 17, wherein the retrieved patient data upon which the risk score for the one or more patients is generated further includes referring physician information and medical examination information that is the subject of the patient appointment, and the generating the risk score includes:
further training the machine-learning component with historical referring physician information and medical examination information that is the subject of the historical patient appointments.

19. The non-transitory computer readable medium of claim 16, wherein the machine learning component comprises an Extreme Gradient Boost (XGB) classifier or a Lasso Logistic algorithm.

20. The non-transitory computer readable medium of claim 1, wherein the risk score for the one or more patients is generated at a fixed time before the medical appointment of the one or more patients.

21. The non-transitory computer readable medium of claim 1, wherein the method further includes:
dynamically computing the risk score using changes in one or more of patient information, weather information, traffic information, transportation information, and/or social events information.

22. A non-transitory computer readable medium storing instructions executable by at least one electronic processor to patient appointment notification method, the method comprising:
training a machine-learning (ML) component with historical training data including at least historical patient data including historical patient histories of no shows or cancellations;
accessing a scheduler of a medical facility to identify patients scheduled for medical appointments;
retrieving patient data for the patients from one or more databases;
generating a risk score for the patients based on at least the retrieved patient data and the trained ML component, the risk score being indicative of a likelihood that one or more of the patients will no show or late cancel the medical appointment; and
for patients having a risk score exceeding a predetermined risk score threshold, generating a notification based at least on the risk score exceeding a predetermined risk score threshold and outputting the notification to a device.
